(19) United States
(12) Patent Application Publication
(10) Pub. No.: US 2008/0166426 A1
(43) Pub. Date: Jul. 10, 2008

(54) ANITBACTERIAL COMPOSITIONS AND METHODS OF TREATMENT

(76) Inventor: Gary Pekoe, Virginia Beach, VA (US)

Correspondence Address: WILLIAMS MULLEN, 222 CENTRAL PARK AVENUE, SUITE 1700, VIRGINIA BEACH, VA, 23462, US

(21) Appl. No.: 12/022,627

(22) Filed: Jan. 30, 2008

Related U.S. Application Data
(63) Continuation-in-part of application No. 11/078,987, filed on Mar. 11, 2005.

(51) Int. Cl. A61K 36/752; A61K 36/18; A61P 31/04; A61K 33/32

(52) U.S. Cl. 424/641; 424/769; 424/736

(57) ABSTRACT

A composition and method of treatment for the Staphylococci, Streptococci, and Enterococcus faecalis Family of infections and their associated symptoms using Sangre de Grado (“SDG”). SDG is extracted from the lechleri Croton tree. SDG is formulated and administered to treat the Staphylococci, Streptococci, and Enterococcus faecalis Family of infections and their associated symptoms. SDG is effective against these various bacterial infections including the often deadly MRSA.

### RELATED APPLICATION DATA

This application is a continuation-in-part of application Ser. No. 11/078,987 filed Mar. 11, 2005 which is incorporated by reference in its entirety.

### FIELD OF THE INVENTION

The invention relates to a composition and method of treatment for the Staphylococci, Streptococci, and Enterococcus faecalis Family of infections and their associated symptoms using Sangre de Grado, an extract from the lechleri Croton tree.

### BACKGROUND OF THE INVENTION

Bacterial infections are a problem across the world, causing many of the infections that afflict individuals today. Staphylococcus bacteria can cause many types of infections through direct infection or indirectly through toxins the bacteria release. Methlicillin-resistant Staphylococcus (“MRSA”) is the newly feared “super-bug” of the Staphylococcus family because of its resistance to most antibiotics. It is a rapidly evolving and sometimes fatal infection that afflicts around 1.2 million people each year in the United States. Another common bacterial family that is sometimes difficult to combat is Streptococcus, which can cause mild to severe infections in humans. Strep throat is the most common infection caused by this type of bacterial and affects 10 million people every year. New treatments for Staphylococci, Streptococci, and other bacterial infections are needed, in particular as drug resistance increases.

### SUMMARY OF THE INVENTION

MRSA infection is caused by Staphylococcus aureus bacteria; often called “staph.” Decades ago, a strain of staph emerged that was resistant to the broad-spectrum antibiotics commonly used. MRSA was one of the first germs to outwit all but the most powerful drug therapies.

Staph bacteria are normally found on the skin or in the nose of about one-third of the population. Staph bacteria are generally harmless unless they enter the body through a cut or other wound, and even then they often cause only minor skin problems in healthy people. But in older adults, children, and people who are ill or have weakened immune systems ordinary staph infections can cause serious illness. If left untreated, or if treated with MRSA-resistant antibiotics, MRSA infections can be fatal.

The present invention provides a composition and method of treatment for the Staphylococci, Streptococci, and Enterococcus faecalis Family of infections and their associated symptoms using Sangre de Grado (“SDG”). SDG is a red viscous sap and/or extract from the lechleri Croton tree. SDG has been previously shown to treat various viral infections and their associated symptoms such as but not limited to smallpox, cowpox, and Molluscum contagiosum. Of course, effectiveness against a viral strain provides no indication of activity against a bacterial strain. Neither does a positive effect against one bacterial strain indicate effectiveness against another bacterial strain, particularly when a bacterial strain has shown resistance to other front-line drugs. Now there is surprising confirmation that SDG is effective against various bacterial infections including the antibiotic resistant and often deadly MRSA.

### DETAIL DESCRIPTION OF INVENTION

SDG is a red viscous sap and/or extract from the lechleri Croton tree. Sap is also referred to as latex and or resin. The SDG extract can be made from any or all parts of the lechleri Croton tree such as but not limited to sap, bark, roots, stems, and/or leaves. SDG is also known as Dragon's Blood, Sangre de Drado, Drago, Sangtie de Drago, Sangue de Agua Products derived from the Croton tree species have been traded and used for centuries. These products have included therapeutic treatments, food additives, and natural medicines. Croton tree species include Croton salutaris, Croton gossypifolius, Croton palanostima, Croton erythrochilus, Croton lechleri, and Croton draconoides.

SDG from Croton lechleri was tested against the following bacterial species:

| | |
|---|---|
| Streptococcus pneumoniae (ATCC 49619) | (SPNEU) |
| Streptococcus pyogenes (clinical isolate) | (GABHS) |
| Enterococcus faecalis (ATCC 29212) | (EFAECL) |
| Coagulase-negative Staphylococcus (clinical isolate) | (CNS) |
| Escherichia coli (ATCC 25922) | (EC) |
| Pseudomonas aeruginosa (ATCC 27853) | (PAER) |
| Klebsiella pneumoniae (ATCC 700603) | (KPNEU) |
| Staphylococcus aureus (ATCC 52923) | (SAUR) |
| Methicillin-resistant Staphylococcus aureus (ATCC 49476) | (MRSA) |

Isolates were subcultured the day before testing. On the day of testing, a bacterial suspension (0.08-0.12 MacFarland units) of each isolate was made in Trypticase Soy Broth (TSB), and these suspensions were used to plate a lawn of organisms on Mueller-Hinton agar or Mueller-Hinton sheeps blood agar. A control antibiotic Kirby-Bauer disk plus K-B disks containing 25 uL of SDG dilutions were applied to these plates, and the plates were incubated overnight. The next day, the width of the zone of growth inhibition surrounding each K-B disk was recorded (in mm).

SDG was used neat (SDG⁰) and in 10-fold dilutions (SDG⁻¹, SDG⁻², SDG⁻³, SDG⁻⁴) made in dimethylsulfoxide (DMSO). Control experiments showed that neat SDG precipitated when mixed into either TSB or Hanks Balanced salt solution with Ca⁺⁺ and Mg⁺, but was apparently soluble in DMSO, ethanol or methanol. When initially diluted into DMSO, EtOH or MeOH at 1:10 and subsequently diluted into TSB, SDG continued to precipitate. Therefore, all dilutions were made into DMSO and a K-B disk containing 25 uL DMSO was included in all experiments as a vehicle control.

Neat SDG and SDG dilutions in DMSO were stored on the bench-top at room temperature. To load K-B disks, dry blank K-B disks were lined up on Parafilm, and 25 uL of the appropriate material was placed on the disk and allowed to soak in. Decreasingly intense brown discoloration of the K-B disks was obvious at SDG⁰-SDG⁻² concentrations. The wet disks were then transferred to the plated bacterial lawns using a sterile needle.

After overnight incubation, a brown precipitate was present on the surface of the agar in the immediate surrounding area of the SDG⁰ disks, and a transparent brown discoloration of the agar was visible around the SDG⁰ and SDG⁻¹ disks. The precipitation around the SDG⁰ K-B disk could make clear identification of the zone around this disk difficult if the zone was small. With the KPNEU isolate, there was the distinct impression that colonies growing up close to the SDG⁰ disk became brown pigmented. Results are shown in the table below.

| | Antibiotic | DMSO | SDG⁰ | SDG⁻¹ | SDG⁻² | SDG⁻³ | SDG⁻⁴ |
|---|---|---|---|---|---|---|---|
| SPNEU | | | | | | | |
| Jun. 3, 2005 | AMP 14 | 0 | 2 | 1 | <1 | <1 | <1 |
| Jun. 6, 2005 | AMP 13 | 1 | 4 | 1 | <1 | <1 | <1 |
| Jun. 7, 2005 | AMPno growth | | | | | | |
| GABHS | | | | | | | |
| Jun. 3, 2005 | AMP 13 | 0 | 2 | 1 | <1 | <1 | <1 |
| Jun. 6, 2005 | AMP 16 | <1 | 1 | <1 | Err | Err | Err |
| Jun. 7, 2005 | AMP 13 | <1 | 2 | 1 | 1 | <1 | <1 |
| EFAECL | | | | | | | |
| Jun. 3, 2005 | AMP 8 | 0 | 3 | 2 | 1 | <1 | <1 |
| Jun. 6, 2005 | GM 5 | <1 | 2 | 1 | <1 | <1 | <1 |
| Jun. 7, 2005 | AMP 8 | <1 | 3 | 1 | <1 | <1 | <1 |
| CNS | | | | | | | |
| Jun. 6, 2005 | GM 11 | <1 | 4 | 4 | 1 | <1 | <1 |
| Jun. 7, 2005 | GM 13 | <1 | 5 | 4 | 1 | <1 | <1 |
| EC | | | | | | | |
| Jun. 3, 2005 | GM 9 | 0 | <1 | 4 | 2 | <1 | <1 |
| Jun. 6, 2005 | GM 8 | <1 | ppt | 5 | 1 | <1 | <1 |
| Jun. 7, 2005 | GM 7 | <1 | <1 | 1 | <1 | <1 | <1 |
| PAER | | | | | | | |
| Jun. 3, 2005 | GM 7 | 0 | 4 | 1 | 1 | <1 | <1 |
| Jun. 6, 2005 | GM 10 | <1 | 1 | 1 | <1 | <1 | <1 |
| Jun. 7, 2005 | GM 8 | <1 | <1 | <1 | <1 | <1 | <1 |
| KPNEU | | | | | | | |
| Jun. 3, 2005 | GM 4 | 0 | <1 | <1 | <1 | <1 | <1 |
| Jun. 6, 2005 | GM 4 | <1 | <1 | <1 | <1 | <1 | <1 |
| Jun. 7, 2005 | GM 5 | <1 | <1 | <1 | <1 | <1 | <1 |
| SAUR | | | | | | | |
| Jun. 2, 2005 | GM 6 | 0 | 3 | 2 | <1 | <1 | <1 |
| Jun. 3, 2005 | GM 7 | 0 | 3 | 2 | <1 | <1 | <1 |
| Jun. 6, 2005 | GM 8 | <1 | 3 | 3 | 1 | <1 | <1 |
| Jun. 7, 2005 | GM 8 | 0 | 4 | 4 | <1 | <1 | <1 |
| MRSA | | | | | | | |
| Jun. 3, 2005 | GM 1 | 0 | 5 | 3 | <1 | <1 | <1 |
| Jun. 6, 2005 | GM <1 | <1 | 4 | 4 | <1 | <1 | <1 |
| Jun. 7, 2005 | GM 1 | 0 | 5 | 4 | <1 | <1 | <1 |

The results indicate that SDG had the greatest activity against the following gram positive organisms: SPNEU, EFAECL, CNS, SAUR and MRSA. These organisms can be categorized into three groups: Staphylococci, Streptococci, and Enterococcus faecalis. SDG activity against Gram negative rods was found to be variably weak. SDG was insoluble in aqueous buffer which likely limited its diffusion into agar. Thus SDG's actual antibacterial activity may be greater than what seen in these experiments. However, this insolubility could potentially limit SDG's applicability in biological systems.

The results indicate that SDG is effective against Staphylococci, Streptococci, and Enterococcus faecalis. Results further indicate that SDG should also be tested for activity against other related strains of Staphylococcus as well as related gram positive bacteria. Future research should focus on ways to incorporate this product into medically related supplies due to SDG's activity against Staphylococcus aureus and MRSA, as well as smallpox and cowpox (reported in a previous study). SDG is one of only a very few number of drugs which may treat MRSA, thus this product has a large potential use in the medical field across the world.

Thus it will be appreciated that the composition in accord with the invention, for use in treating the Staphylococci, Streptococci, and Enterococcus faecalis Family of infections and their associated symptoms such as but not limited to bacterial induced lesions, is comprised of SDG or one or more individual components of SDG and includes synthesized analogs thereof. SDG is obtained from Croton lechleri trees. All parts of the tree may be used such as but not limited to sap, resin, bark, roots, stems, and/or leaves. In one embodiment, sap or resin from the tree is used to make compositions of the invention and practice methods of the invention. In another embodiment an extract is made from various parts of the tree such as but not limited to the sap, resin, bark, roots, stems, and/or leaves.

The SDG composition of the present invention may be alternatively obtained from any of a number of tree species within the genus Croton (family Euphorbiaceae) that grow in Central and South America such as but not limited to the Amazon region of Peru, Ecuador, Brazil, Dominican Republic, Mexico and Colombia. Other Croton species may also be used as a source for SDG. Other Croton species include Croton salutaris, Croton gossypifolius, Croton palanostima, Croton erythrochilus, Croton urucurana, Croton xalapensis and Croton draconoides. The composition may be alternately derived from one tree, multiple trees of the same species, or from multiple trees of different species.

Tree specimens of different sizes and ages can be used. In some embodiments, the tree specimen is a tree of sufficient age to produce harvestable sap. In one embodiment of the present invention, the source tree is approximately thirty inches in average diameter and approximately sixty feet high.

In one embodiment of the present invention, sap from the Croton lechleri tree may be collected to manufacture the therapeutic composition. One first locates an appropriate tree for harvesting. The typical tree is fast growing, reaching heights of 30-45 feet in 3 years. The sap can be harvested like rubber (at a slower rate). Repeated tapping of the tree can lead to excessive scar damage and fungal infections in the tree. This diminishes productivity. The trees can be harvested at 2-3 years of age. After a tree has fallen, the branches and trunk are cut into smaller segments and the bark is lacerated to allow the resin to escape. These segments are then stacked on collecting sheets to collect the resin as the stack “bleeds.”

The sap can also be collected from living trees. A large collecting sheet, e.g., a 15.times.15 foot tarp, is attached to the “collecting side” of the tree, e.g., using string, duct tape, or anther fastener. The tree is wounded up to about 5 inches deep, and more preferably about 0.5 to 1.5 inches deep, e.g., with a machete, with lengthy slices all up and down the collecting side of the tree (over the tarp), and the tree bleeds (sap falls) for approximately 90 minutes onto the tarp.

Using gloves and a medical facial mask to prevent contamination, the larger debris is removed from the sap. The sap is next transferred from the collecting tarp or container into a sterile (e.g., FDA standard laboratory/medical) container, which is then capped. For example, the tarp is folded and the sap is channeled along the fold into the sterile container. In a preferred variation, the collected sap is micro filtered, e.g., to 30 microns, and bottled for storage and sale. Step filtrations of 100.mu.m and 30.mu.m may be used. The product is then packaged or bottled using current Good Manufacturing Practices (GMPs) and Standard Operating Procedures (SOPs) for all processes. During all phases of production and packaging, strict quality control and safety standards are emphasized. Spectrometry and/or other methods may be used to test consistency from multiple collections from a source tree or collect from different trees. In one embodiment of the present invention, irradiation, chemicals, heat, or other means are used to sterilize the composition.

Analysis of the sap from an appropriate Croton tree source has revealed a composition comprising compounds such as but not limited to Cyanidole (flavonolmonomers): (+)-gallocatechin, (+)-catechin, (−)-epicatechin, (−)-epigallocatechin, proanthocyanidin(proanthocyanidindimerB-4, proanthocyanidintrimer, proanthocyanidintetramer, proanthocyanidinheptamer), cedrucine, Daucosterol, Dihydrobenzofuran, Dimethylcedrusine, Isoboldine, korberinA&B, magnoflorine, norisoboldine, procyanidins, resin, tannin, hardwickiicacid, bincatriole, crolechinole, crolechinicacid, coberineA, coberineB, taspine, dihyhrobenzofuranlignans; -3x,4-O-dimethylocedrusine, -4-O-methylocedrusine, 1,3,5-trimethoxybenzene, 2,4,6-trimethoxyphenol, 4-hydroxyphenethylalcohol, beta-sitosterol, beta-sitosterol-beta-D-, glucopyranoside, beta-Pinene, Betaine, Borneol, Calamenene, Camphene, Cuparophenol, D-Limonen, Dipentene, EO, Eugenol, Euparophenol, alpha-calacorene, alpha-copaene, alpha-pinene, alpha-thujone, beta-Caryophyllene, beta-Elemene, Gamma-Terpinene, Gamma-Terpinole, Lignin, Linalool, Methylthymol, Myrcene, Para-cymene(p-cymene), Pectic-acid, Terpinene-4-ol, Vanillin, piridona, aporfina, quinoleina, and the SP-303 (including: alkaloids, proanthocyanidins (antioxidants), terpines, diterpenes, phenols, tannins, andlignans) simplephenols, phytosterols, and biologically active alkaloids.

Another analysis of molecules derived from a Croton tree and that may be present in the crude compositions of the present invention are listed in the following table:

Presence of Compounds in Croton Lechleri (Euphorbiaceae)

| Compound | Type | Source |
|---|---|---|
| BENZENE, 1-3-5 TRIMETHOXY | BENZENOID | BARK |
| | | SAP |
| | | SAP |
| BENZOFURAN-5-YL, 2-3-DIHYDRO: 2- | LIGNAN | SAP |
| (3-4-DIMETHOXY-PHENYL): 7- | | |
| METHOXY-3-METHOXY-CARBONYL- | | |
| PROPAN-1-OIC ACID METHYL ESTER | | |
| BENZOFURAN-5-YL, 2-3-DIHYDRO: 2- | LIGNAN | SAP |
| (3-4-DIMETHOXY-PHENYL): 7- | | |
| METHOXY-3-METHOXY-CARBONYL- | | |
| PROPEN-1-OIC-ACID METHYL ESTER | | |
| BENZOFURAN-5-YL, 2-3-DEHYDRO: 2- | LIGNAN | SAP |
| (4-HYDROXY-3-METHOXY-PHENYL)- | | |
| 7-METHOXY-3-METHOXY- | | |
| CARBONYL-PROPEN-1-OIC ACID | | |
| METHYL ESTER | | |
| BENZYL ALCOHOL, 3-4-DIMETHOXY | BENZENOID | BARK |
| | | SAP |
| BINCATRIOL | DITERPENE | BARK |
| | | SAP |
| BOLDINE, ISO | ISOQUINOLINE | LEAF |
| | ALKALOID | |
| BOLDINE, ISO: NOR: | ISOQUINOLINE | LEAF |
| | ALKALOID | |
| CATECHIN(4-ALPHA-8)- | FLAVONOID | LATEX |
| GALLOCATECHIN(4-ALPHA-6)- | | (UNSPEC PART) |
| GALLOCATECHIN | | |
| CATECHIN(4-ALPHA-8)- | FLAVONOID | LATEX |
| GALLOCATECHIN(4-ALPHA-8)- | | (UNSPEC PART) |
| GALLOCATECHIN, (+): | | |
| CATECHIN, (+): | FLAVONOID | LATEX |
| | | (UNSPEC PART) |
| | | LATEX |
| | | (UNSPEC PART) |
| | | SAP |
| CATECHIN, EPI: (−): | FLAVONOID | LATEX |
| | | (UNSPEC PART) |
| | | LATEX |
| | | (UNSPEC PART) |
| | | SAP |
| CEDRUSIN, 3′-4-0-DIMETHYL: | LIGNAN | SAP |
| | | SAP |
| CEDRUSIN, 3′-4-0-DIMETHYL: (DL): | LIGNAN | SAP |
| CEDRUSIN, 4-0-METHYL: | LIGNAN | SAP |
| CROLECHINIC ACID | DITERPENE | BARK |
| | | SAP |
| | | SAP |
| CROLECHINOL | DITERPENE | SAP |
| | | BARK |
| DAUSCOSTEROL | STEROID | BARK |
| | | SAP |
| | | SAP |
| GALLOCATECHIN(4-ALPHA-6)-EPI- | FLAVONOID | LATEX |
| GALLOCATECHIN | | (UNSPEC PART) |
| GALLOCATECHIN(4-ALPHA-8)-EPI- | FLAVONOID | LATEX |
| CATECHIN | | (UNSPEC PART) |
| GALLOCATECHIN(4-ALPHA-8)- | FLAVONOID | LATEX |
| GALLOCATECHIN(4-ALPHA-8)-EPI- | | (UNSPEC PART) |
| GALLOCATECHIN | | |
| GALLOCATECHIN(4-ALPHA-8)- | FLAVONOID | LATEX |
| GALLOCATECHIN-(4-ALPHA-8)-EPI- | | (UNSPEC PART) |
| GALLOCATECHIN | | |
| GALLOCATECHIN, (+): | FLAVONOID | LATEX |
| | | (UNSPEC PART) |
| | | SAP |
| GALLOCATECHIN, EPI: (−): | FLAVONOID | LATEX |
| | | (UNSPEC PART) |
| | | LATEX |
| | | (UNSPEC PART) |
| | | SAP |
| GLAUCINE | ISOQUINOLINE | LEAF |
| | ALKALOID | |
| HARDWICKIIC ACID | DITERPENE | BARK |
| | | SAP |
| KORBERIN A | DITERPENE | BARK |
| | | SAP |
| KORBERIN B | DITERPENE | BARK |
| | | SAP |
| MAGNOFLORINE | ISOQUINOLINE | LEAF |
| | ALKALOID | |
| PHENETHYL ALCOHOL, 4- | BENZENOID | SAP |
| HYDROXY: | | BARK |
| | | SAP |
| PHENETHYL ALCOHOL, 4- | BENZENOID | BARK |
| HYDROXY: ACETATE | | |
| PHENOL, 2-4-6-TRIMETHOXY: | BENZENOID | SAP |
| | | BARK |
| | | SAP |
| PHENOL, 3-4-DIMETHOXY: | BENZENOID | BARK |
| | | SAP |
| PROCYANIDIN B-1 | FLAVONOID | LATEX |
| | | (UNSPEC PART) |
| | | LATEX |
| | | (UNSPEC PART) |
| PROCYANIDIN B-2 | FLAVONOID | LATEX |
| | | (UNSPEC PART) |
| PROCYANIDIN B-4 | FLAVONOID | LATEX |
| | | (UNSPEC PART) |
| | | SAP |
| SINOACUTINE | ISOQUINOLINE | LEAF |
| | ALKALOID | |
| SITOSTENONE, BETA: | STEROID | BARK |
| | | SAP |
| SITOSTEROL, BETA: | STEROID | SAP |
| | | BARK |
| | | SAP |
| SP-303 | FLAVONOID | LATEX |
| | | (UNSPEC PART) |
| TASPINE | ALKALOID | SAP |
| | | LATEX |
| | | (UNSPEC PART) |
| | | SAP |
| | | LATEX (STEM) |
| | | SAP |
| | | LATEX |
| | | (UNSPEC PART) |
| | | LATEX |
| | | (UNSPEC PART) |
| | | SAP |
| | | LEAF |
| | | BARK |
| | | SAP |
| THALIPORPHINE | ISOQUINOLINE | LEAF |
| | ALKALOID | |

In one embodiment of the present invention, compositions comprising one or more DG components through artificial manufacture may be used in the place of a naturally-SDG components and composition, wherein the artificial components and compositions mate the activity or have substantially the same activity of the natural composition.

In one embodiment of the present invention, the composition comprises SDG and a pharmaceutically acceptable diluent, adjuvant, excipient, or carrier, to facilitate and/or improve administration to a subject. Pharmaceutical formulation chemistry to create biologically active compounds and analogues can be used directly to practice materials and methods of the invention. This is a well developed art, and exemplary formulation materials and methods are discussed below.

In support of the invention, zinc functions as an antioxidant within the human body. Zinc is capable of protecting proteins and enzymes against free radical attack, or oxidation. Free radicals are unstable, reactive molecules that damage the normal functions of substances, such as cells, via rapid reactions. Zinc acts as an antioxidant to protect specific regions of enzymes from free radical attack. Through this action, zinc preserves an enzymes activity and stability. Therefore in one embodiment of the present invention, zinc is added to the composition. In one embodiment, zinc gluconate, or zinc sulfate is used within a range of about 10-100 mMol. In another embodiment of the present invention, zinc gluconate, or zinc sulfate is used within a range of about 25-75 mMol.

Further in support of the present invention, citrus extract, such as but not limited to orange, lemon, lime, grapefruit, and combinations thereof are used. As used herein the term “citrus extract” refers to a solution or preparation containing the active principles of a plant, tree, fruit or the like and can be made from any or all parts of the plant, tree, fruit, or the like such as but not limited to sap, bark, roots, stems, fruit and/or leaves. Specifically, citrus extracts also function as antioxidants by reducing the amount of oxidative stress experienced in the body or individual cell. The function of citrus extract is similar to that of zinc. Therefore in another embodiment of the present invention, citrus extract is added to the composition in a range of 0.5-5% W/V.

In yet another embodiment of the present invention, zinc and citrus extract are both added to the composition within a range of about 10-100 mMol and 0.5-5% W/V respectively.

The phrase “pharmaceutically” or “pharmacologically acceptable” refers to molecular entities and compositions that do not produce adverse, allergic, or other untoward reactions when administered to a subject, e.g., topically, transdermally, parenterally, by inhalation spray, vaginally, rectally, by eye drop, or by intracranial injection. The term parenteral as used herein includes subcutaneous injections, intravenous, intramuscular, intracisternal injection, or infusion techniques. Administration by intravenous, intradermal, intramuscular, intramammary, intraperitoneal, intrathecal, retrobulbar, intrapulmonary injection and/or surgical imtreeation at a particular site is contemplated as well.) Generally, this will also entail preparing compositions that are essentially free of pyrogens, as well as other impurities that could be harmful to humans or animals. The term pharmaceutically acceptable carrier includes any and all solvents, dispersion media, coatings, antibacterial and antifungal agents, isotonic and absorption delaying agents and the like. The use of such media and agents for pharmaceutically active substances is well known in the art. Topical carriers include creams, salves, foams, lotions, collagen preparations, gels, and ointments.

The SDG composition may include acid or base components. As used herein, the term “component” is intended to include those naturally occurring compounds and molecules identified in SDG as well as those formulated such as but not limited to pharmaceutically acceptable salts, esters, and combinations thereof.

For example, when an acidic substituent, such as —COOH, is present, the ammonium, sodium, potassium, calcium and like salts, are contemplated as possible embodiments for administration to a biological host. When a basic group such as amino or a basic heteroaryl radical, such as pyridyl is present, then an acidic salt, such as hydrochloride, hydrobromide, acetate, maleate, palmoate, phosphate, methanesulfonate, p-toluenesulfonate, and the like, is contemplated as a possible form for administration to a biological host.

Similarly, where an acid group is present, then pharmaceutically acceptable esters of the compound e.g., methyl, tert-butyl, pivaloyloxymethyl, succinyl, and the like are contemplated as possible forms of the compounds, such esters being known in the art for modifying solubility and/or hydrolysis characteristics for use as sustained release or prodrug formulations. In addition, some components may form solvates with water or common organic solvents. Such solvates are contemplated as part of the present invention as well.

Aqueous suspensions may contain the SDG composition or active compounds in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the SDG or components of it in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Dispersible powders and granules suitable for preparation of an aqueous suspension by the addition of water provide the active composition admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

One embodiment of the composition also may be formulated as a dispersable powder for dusting the skin, hair, fur, or feathers of humans or animals. One embodiment of the composition may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. Emulsions may also contain sweetening and flavoring agents and scent enhancers.

One embodiment of the composition may also be in the form of suppositories for rectal administration of the composition. These compositions can be prepared for example by mixing the composition with a suitable non-irritating excipient that is solid at ordinary temperatures but liquid at the rectal temperature and will therefore melt in the rectum to release the drug. Such materials are cocoa butter and polyethylene glycols, for example.

Therapeutic formulations of the compositions useful for practicing the present invention may be prepared for storage by mixing the selected composition having the desired degree of purity with optional physiologically pharmaceutically-acceptable carriers, excipients, or stabilizers (Remington's Pharmaceutical Sciences, 18th edition, A. R. Gennaro, ed., Mack Publishing Company (1990)) in the form of a lyophilized cake or an aqueous solution. Acceptable carriers, excipients or stabilizers are nontoxic to recipients and may be inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics or polyethylene glycol (PEG).

One embodiment of the composition to be used for in vivo administration may be sterile. This is readily accomplished by filtration through sterile filtration membranes, prior to or following lyophilization and reconstitution. One embodiment of the composition for parenteral administration ordinarily will be stored in lyophilized form or in solution.

One embodiment of the composition may be placed into a container having a sterile access port, for example, an intravenous solution bag or vial having a stopper pierceable by a hypodermic injection needle. The route of administration of the composition is in accord with known methods, e.g. topical, or by sustained release systems or imtreeation device.

Suitable examples of sustained-release preparations include semipermeable polymer matrices in the form of shaped articles, e.g. films, or microcapsules. Sustained release matrices include polyesters, hydrogels, polylactides (U.S. Pat. No. 3,773,919, EP 58,481), copolymers of L-glutamic acid and gamma ethyl-L-glutamate (Sidman, et al., Biopolymers, 22: 547-556 (1983)), poly(2-hydroxyethyl-methacrylate) (Langer, et al., J. Biomed. Mater. Res., 15:167-277 (1981) and Langer, Chem. Tech., 12:98-105 (1982)), ethylene vinyl acetate (Langer, et al., supra) or poly-D(−)-3-hydroxybutyric acid (EP 133,988). Sustained-release compositions also may include liposomes, which can be prepared by any of several methods known in the art (e.g., DE 3,218,121; Epstein, et al., Proc. Natl. Acad. Sci. USA, 82:3688-3692 (1985); Hwang, et al., Proc. Natl. Acad. Sci. USA, 77:4030-4034 (1980); EP 52,322; EP 36,676; EP 88,046; EP 143,949).

In one embodiment of the present invention, the SDG composition is a topical composition. In one embodiment, the topical composition is formulated as a cream, a gel, an emollient, a salve, a liquid spray, an aerosol, or an impregnated bandage. In another embodiment, the topical formulation comprises a compound to improve the fragrance of the composition, including but not limited to orange extract or mint extract. In other embodiments, the composition is stored at room temperature in a product bottle, lid firmly closed, for up to one year.

### Dose and Dosing

Undiluted SDG has proven empirically to be very safe and well tolerated in humans whether taken orally or used topically. Thus the frequency of application or administration to a subject can be adjusted upwardly to achieve a desired therapeutic effect. Subjects experiencing side effects should reduce dosage or discontinue use. Depending on the route of administration, a suitable dose may be calculated according to body weight, body surface areas or organ size. Further refinement of the calculations to determine the appropriate treatment dose is routinely made as part of any medical treatment regimen, in view of the pharmacokinetic data observed in animals and/or human clinical trials. Dosage consideration may also be guided by pharmaceutical references, see, e.g., Physician's Desk Reference (Montvale, N.J.), which is incorporated by reference in its entirety. Dosing in humans may also be extrapolated from animal dosages, toxicity studies, and pharmacokinetics, according to standard pharmacological methodologies.

In one embodiment, the SDG composition is applied via dropper onto bacterial-associated lesions and allowed to dry, or rubbed in gently. In another embodiment, the SDG composition is formulated into a cream or gel formulation and is applied on the affected area as a thin drop, and rubbed in gently. In one embodiment of the present invention, it is contemplated that the topical formulation is applied once a day up to once every 15 minutes to the affected areas.

SDG has been used orally by South American natives for the treatment of diarrhea, nausea, vomiting and other stomach ailments. It is has also been taken internally as a tonic, either diluted in water or brewed as a tea. It is also administered to domestic animals that exhibit gastrointestinal symptoms such as but not limited to vomiting, GI distress. Among other things, empirical data attests to the safety of the material, as untoward side effects have not been attributed to its ingestion.

The therapeutic composition may be administered for any length of time, and if necessary may be administered as long as the symptoms, disease, or disorder remains in the subject. Dosages may also be varied during the course of treatment. For example, the dosages may be adjusted if the subject encounters side effects, develops unrelated complications, and/or has a change in the kind, dosage, and/or administration of one or more medications other than those of the combination therapy.

Administration to a subject of the SDG therapy may start before, during, or after symptoms or evidence of bacteria infection appear. In some embodiments, the therapy is started as early as immediately, 15 minutes (min)., 30 min., 1 hour(s) (hr.), 1½ hr., 2 hr., 2½ hr., 3 hr., 4 hr., 5 hr., 6 hr., 7 hr., 8 hr., 9 hr., hr., 11 hr., 12 hr., 16 hr., 18 hr., 20 hr., 22 hr., 24 hr., 36 hr., 48 hr., 60 hr., 72 hr., 84 hr., 96, hr., 5 days, 6, days, 10 days, 13 days, 1 week, 2 weeks, three weeks, 4 weeks, 6 weeks, 8 weeks, 10 weeks, 12 weeks, 1 month, 2 months, 3, 4 months, 5 months, 6 months, 7 months, 8 months, 9 months, 10 months, 11 months, 12 months, 13 months, 14 months, 15 months, 16 months, 17 months, 18 months, or more, or any intermediate length of time, following evidence of a bacteria infection or episode. In some embodiments, the therapy is continued for, hours, days (e.g., 1, 2, 3, 4, 5, 6, 7, 8, 9, 10, or more), I-10 weeks, 1-12 months, or years, or whenever a persistent virus or its symptoms reappear.

The SDG composition may be administered continuously, every 15 minutes 30 min., 1 hour(s) (hr.), 1½ hr., 2 hr., 2½ hr., 3 hr., 4 hr., 6 hr., 8 hr., 12 hr., 24 hr., 36 hr., 48 hr., 3 days, 4 days, 5 days, 6, days, 1 week, 2 weeks, or frequencies intermediate or less than the foregoing.

In one embodiment dosage is 1-2 drops of the undiluted sap per lesion, twice or more daily with the composition applied to each lesion. Multiple drops are applied to a crop of lesions. The drops are allowed to dry (several minutes) or they are gently rubbed (about 15 seconds) over the lesions until the composition changes to a “creamier” state. It then dries very quickly (several seconds). Rubbing should be gentle to prevent autoinoculation of uninfected tissue.

In one embodiment the composition is first applied to a bandage (e.g., gauze), which is then applied to the lesion. This means of application is particularly useful for difficult regions of the human body (groin, armpit, and eyes). The treated bandage is applied to each lesion. If the bandage is separated from the lesion or if the dressing has been worn for 24 hours, a new, treated bandage should be applied. A new dressing is generally applied every day. In one embodiment, the composition is administered until the symptoms (e.g., skin lesions) disappear, become less pronounced, or problematic side effects occur.

Materials and methods of the invention can be practiced on animals, to treat animal bacterial infections and other skin conditions caused by Staphylococci, Streptococci, and Enterococcus faecalis. Treatment of any domestic pet animal, livestock, zoo animals, circus animals, endangered species, and the like is contemplated.

The SDG composition is administered in an amount effective to palliate the symptoms of the conditions and symptoms caused by Staphylococci, Streptococci, and Enterococcus faecalis. As indicated herein, any form of administration and pharmaceutical composition is contemplated. Those of ordinary skill in the art will readily optimize effective dosages and administration regimens as determined by good medical practice and the clinical condition of the individual subject, taking into account such considerations as therapeutic efficacy, risk of toxicity, and side-effects.

Thus it will be appreciated that a method of treatment using the composition in accord with the invention, for treating the Staphylococci, Streptococci, and Enterococcus faecalis Family of infections and their associated symptoms such as but not limited to bacterial induced lesions, is comprised of identifying a subject infected with a bacteria found in the Staphylococci, Streptococci, and Enterococcus faecalis Family and administrating a therapeutically effective amount of the composition in a therapeutic dose and frequency wherein the composition is SDG or one or more individual components of SDG and includes synthesized analogs thereof.

The invention further encompasses compounds and methods for treating subjects, infected with any member of the Staphylococci, Streptococci, and Enterococcus faecalis Family, including subfamilies and genera discovered, undiscovered, presumed eradicated, created, mutated, or yet to evolve or exist. This invention includes Staphylococci, Streptococci, and Enterococcus faecalis native to humans and Staphylococci, Streptococci, and Enterococcus faecalis native to animals but found in human for the purpose of biologic terrorism and those that are not.

In one aspect, the invention is prophylactic method of treatment for any of the bacteria infections described herein, comprising (a) selecting a subject in need of prophylaxis for the Staphylococci, Streptococci, and Enterococcus faecalis Family bacteria, e.g., by identifying or diagnosing the presence of a bacteria infection in a subject or identifying a risk for infection due to infected members of the subject's family, community, etc; and (b) administering to the subject a composition wherein the composition is SDG or one or more individual components of SDG and includes synthesized analogs thereof. Subjects include humans, zoo mammals, mammals domesticated as pets, livestock, and racing animals, including but not limited to felines, bovines, canines, equines, porcines, dromedaries, and others; and birds, including but not limited to zoo birds, pets, and farm birds, e.g., eagles, hawks, canaries, parrots, chickens, turkeys, ostrich, and emu.

In one embodiment of the present invention, the SDG composition may be arranged in a kit or package by unit dose, to permit co-administration with one or more other therapeutic agents, but the SDG composition and the agent are not in admixture. The term “unit dose” as used herein refers to a quantity sufficient to deliver one treatment to a subject identified as having a bacteria infection or identified as at risk for infection with the Staphylococci, Streptococci, and Enterococcus faecalis Family of bacteria.

In another embodiment, the SDG composition may be arranged in a kit or package by unit dose, to permit co-administration with one or more other therapeutic agents and the SDG composition and the agent are in admixture. In another embodiment, the two components to the kit, package by unit dose are packaged with instructions for administering the agents to a subject for treatment. In another embodiment of the present invention, stabilizers which are generally known in the art are added so that the composition is storage stable at least about 12 months.

As used herein “treating” refers to preventing, curing, and/or ameliorating an infection and/or the symptoms associated with or induced by the Staphylococci, Streptococci, and Enterococcus faecalis Family of bacterial infections including but not limited to the reduction of the lesion number and/or size, and/or duration of lesions on the skin, scalp, mouth, nasal cavity, genitals, and other surfaces and/or duration of the infection.

As used herein, the term “antibacterial activity” refers to the ability of the composition, method, or treatment regimen to reduce the size, extent, severity, and duration of infections, lesions, or the communicability of the Staphylococci, Streptococci, and Enterococcus faecalis Family.

As used herein, the term “administration” refers to the process whereby the composition or method of the invention is introduced to a human or animal, which is the host of a virus in the Staphylococci, Streptococci, and Enterococcus faecalis Family, and is in need of treatment for the infection.

As used herein, the term “therapeutically effective” refers to when a composition or method of the invention is properly administered in vivo to a subject and a measurable beneficial effect occurs. Exemplary beneficial effects are described throughout the application, and include measurable antibacterial effects in conditions where bacterial load can be assayed; a reduction of clinically verifiable and/or patient-reported symptoms, including the reduction, impedance or retardation in the growth of lesions; shrinkage of lesions; reduction in the duration of the symptoms caused by the Staphylococci, Streptococci, and Enterococcus faecalis Family directly or indirectly; or complete resolution or curing of the bacteria infection or outbreak.

As yet another aspect of the invention, it is contemplated that SDG or other compositions of the invention are administered to patients in need of treatment in combination with other therapeutics, such as a second agent which is an antibacterial agent. When given in combination with another agent, the amount of SDG given may be reduced accordingly. Second agents are administered in an amount determined to be safe and effective at ameliorating human disease.

It is contemplated that the antibacterial agents are administered in the same formulation as SDG and given simultaneously. Alternatively, the agents may be administered in a separate formulation and still be administered concurrently with SDG. As used herein, concurrently refers to agents given within 30 minutes of each other. The second agent may also be administered prior to administration of SDG. Prior administration refers to administration of the agent within the range of one week prior to SDG treatment up to 30 minutes before administration of SDG. It is further contemplated that the second agent is administered subsequent to administration of the SDG composition. Subsequent administration is meant to describe administration from 30 minutes after SDG administration up to one week after SDG treatment.

Novel formulations that include the SDG material and a second therapeutic agent are themselves aspects of the invention. Such dual agent formulations or kits, when packaged together but not in admixture, may include pharmaceutically acceptable diluents, carriers, stabilizers, or the like, or delivery agents.

The foregoing description of the invention is not intended to be limiting or comprehensive, and additional embodiments are contemplated. All such embodiments are aspects of the invention. Moreover, for the sake of brevity, various details that are applicable to multiple embodiments have not been repeated for every embodiment. Variations reflecting combinations and rearrangements of the embodiments described herein are intended as aspects of the invention. In addition to the foregoing, the invention includes, as an additional aspect, all embodiments of the invention narrower in scope in any way than the variations specifically mentioned above. For example, for aspects described as a genus or range, such as dosages or dosing regimens, every subgenus, sub-range or species is specifically contemplated as an embodiment of the invention.

Without departing from the scope thereof, one skilled in the art can easily ascertain from the forgoing description the essential characteristics of this invention to adapt it to various uses and conditions. The foregoing description and examples have been set forth merely to illustrate the invention and are not intended to be limiting. Because modifications of the disclosed embodiments incorporating the spirit and substance of the invention may occur to persons skilled in the art, the invention should be construed to include everything within the scope of the appended claims and equivalents thereof. The patents, patent application publications and other publications referenced herein are incorporated in their entirety.

Although the applicant invented the full scope of the claims appended hereto, the claims are not intended to encompass within their scope the prior art work of others. Therefore, in the event that statutory prior art within the scope of a claim is brought to the attention of the applicants by a Patent Office or other entity or individual, the applicant reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

What is claimed:

1. A pharmaceutical composition for treatment of a bacterial infection and its associated symptoms wherein the bacterial infection is selected from the group consisting of Staphylococci, Streptococci, Enterococcus faecalis, and combinations thereof, comprising: pharmaceutically effective amounts of at least one component of Sangre de Grado, wherein Sangre de Grado is isolated from a lechleri Croton tree.

2. The composition of claim 1, further comprising zinc selected from the group consisting of zinc gluconate, zinc sulfate, and combinations thereof.

3. The composition of claim 2, wherein the concentration of zinc is within the range of about 25-75 mMol.

4. The composition of claim 2, wherein the concentration of zinc is within the range of about 10-100 mMol.

5. The composition of claim 1, further comprising citrus extract selected from the group consisting of orange, lemon, lime, grapefruit, and combinations thereof.

6. The composition of claim 5, wherein the concentration of citrus extract is within the range of about 0.5-5% W/V.

7. The composition of claim 1, further comprising:
a) Zinc selected from the group consisting of zinc gluconate, zinc sulfate, and combinations thereof, the zinc within a range of about 10-100 mMol; and
b) Citrus extract selected from the group consisting of orange, lemon, lime, grapefruit, and combinations thereof, the citrus extract within a range of about 0.5-5% W/V.

8. The composition of claim 1, wherein the bacterial infection is Methicillin-resistant Staphylococcus.

9. The composition of claim 1, further comprising a pharmaceutically acceptable carrier.

10. The composition of claim 1, wherein the composition is suitable for topical application.

11. The composition of claim 1, wherein the composition is suitable for ingestion.

12. The composition of claim 1, wherein the composition is suitable for intravenous administration.

13. The composition of claim 1, wherein the composition is storage stable at least about 12 months.

14. A method of treating a bacterial infection, wherein the bacterial infection is selected from the group consisting of Staphylococci, Streptococci, Enterococcus faecalis, and combinations thereof, comprising:
a) Identifying a subject infected with a bacteria found in the Staphylococci, Streptococci, and Enterococcus faecalis Family; and
b) Administering to the subject a pharmaceutical composition comprising: pharmaceutically effective amounts of at least one component of Sangre de Grado, wherein Sangre de Grado is isolated from a lechleri Croton tree.

15. A method of treating a bacterial infection, wherein the bacterial infection is selected from the group consisting of Staphylococci, Streptococci, Enterococcus faecalis, and combinations thereof, comprising:
a) Identifying a subject infected with a bacteria found in the Staphylococci, Streptococci, and Enterococcus faecalis Family; and
b) Administering to the subject a pharmaceutical composition comprising:
i) pharmaceutically effective amounts of at least one component of Sangre de Grado, wherein Sangre de Grado is isolated from a lechleri Croton tree;
ii) zinc selected from the group consisting of zinc gluconate, zinc sulfate, and combinations thereof, the zinc within a range of about 10-100 mMol; and
iii) citrus extract selected from the group consisting of orange, lemon, lime, grapefruit, and combinations thereof, the citrus extract within a range of about 0.5-5% W/V.

16. The method of claim 14, wherein the composition is administered topically.

17. The method of claim 14, wherein the composition is administered orally.

18. The method of claim 14, wherein the composition is administered intravenously.

19. A kit for treatment of a bacterial infection and its associated symptoms wherein the bacterial infection is selected from the group consisting of Staphylococci, Streptococci, Enterococcus faecalis, and combinations thereof, comprising at least one unit dose of pharmaceutically effective amounts of at least one component of Sangre de Grado, wherein Sangre de Grado is isolated from a lechleri Croton tree.

20. A kit for treatment of a bacterial infection and its associated symptoms wherein the bacterial infection is selected from the group consisting of Staphylococci, Streptococci, Enterococcus faecalis, and combinations thereof, comprising:
a) At least one unit dose of pharmaceutically effective amounts of at least one component of Sangre de Grado, wherein Sangre de Grado is isolated from a lechleri Croton tree;
b) Zinc selected from the group consisting of zinc gluconate, zinc sulfate and combinations thereof, the zinc within a range of about 10-100 mMol; and
c) Citrus extract selected from the group consisting of orange, lemon, lime, grapefruit and combinations thereof, the citrus extract within a range of about 0.5-5% W/V.
